(19) United States
(12) Patent Application Publication
(10) Pub. No.: US 2022/0071294 A1
(43) Pub. Date: Mar. 10, 2022

(54) SMART VAPORIZER

(71) Applicant: Radient Technologies Innovations Inc., Edmonton (CA)

(72) Inventors: Michael Cabigon, Edmonton (CA); Jim Seethram, Edmonton (CA); Steven Splinter, North Vancouver (CA); Denis Taschuk, Edmonton (CA)

(21) Appl. No.: 17/238,040

(22) Filed: Apr. 22, 2021

Related U.S. Application Data
(63) Continuation of application No. PCT/IB2019/058744, filed on Oct. 14, 2019.
(60) Provisional application No. 62/749,034, filed on Oct. 22, 2018.

(51) Int. Cl. A24F 40/53; G05B 15/02; A24F 40/65; A24F 40/57; A24F 40/51; A24F 40/485; A24F 40/10

(52) U.S. Cl. CPC A24F 40/53; G05B 15/02; A24F 40/65; A24F 40/10; A24F 40/51; A24F 40/485; A24F 40/57

(57) ABSTRACT

The present disclosure is directed to apparatus, methods, and non-transitory computer readable storage media that may control the operation of vaporizing cannabinoids included in a vaporizer cartridge. Apparatus and methods consistent with the present disclosure may identify contents included in a vape cartridge based on an identifier that may be scanned or read. Once the materials in a cartridge are identified, a smart vaporizer may be configured to vaporize those materials in controlled ways. In certain instances, recommended vaporization parameters may be modified based on user input. Parameters that control the heating of a heating element or the opening of a valve that controls a flow of liquid from a reservoir at the cartridge may be changed based on environmental conditions such that a user using the smart vaporizer may be able to inhale vaporized substances at preferred temperatures or flow rates even when environmental conditions change.

### CROSS-REFERENCE TO RELATED APPLICATIONS

The application is a continuation of PCT/IB2019/058744 which claims priority benefit of U.S. provisional patent application No. 62/749,034, filed on Oct. 22, 2018, the disclosures of which are incorporated, herein, by reference.

### BACKGROUND OF THE INVENTION

### 1. Field of Invention

The present disclosure is generally related to vaporization devices. More specifically the present disclosure is directed to cartridge-specific vaporization in accordance with vaporization rules or settings.

### 2. Description of the Related Art

A vaporizer or vaporization device (“vape”) is a device designed to convert concentrated liquids—including cannabis-based extracts and liquids—to an inhalant vapor or aerosol. Such liquids may be provided by attachable cartridges to the vape device. As such, the cartridge-provided liquids—which may include cannabinoids and other compounds—may be heated within a vape device to a temperature by a heating element that vaporizes the cannabinoids, terpenes, terpenoids, flavonoids, and/or other aromatic or taste-enhancing agents.

Currently, there are a variety of portable and desktop vaporizers available for purchase. Each of these different vaporizers use different types of heating mechanisms or wicks that provide for a flow of a cannabinoid-containing liquid to the heating mechanism such that cannabinoids and other compounds can be vaporized and provided to a user. The cannabinoids and other components of the vape liquids may have different vaporization temperatures, as well as different temperatures at which the compounds may be degraded or destroyed. For example, the psychoactive cannabinoid tetrahydrocannabinol (THC) starts to vaporize around 157 degrees ° C. or 315 degrees Fahrenheit (° F.), while the non-psychoactive cannabinoid cannabidiol (CBD) may vaporize across a range of temperatures from about 160 to 180° C. or 320-356° F., depending on the overall composition of the plant. Other cannabinoids such as tetrahydrocannabivarin (THCV) may require even higher temperatures.

It may be possible to modulate the effects during the vaping of cannabis-based liquids by modulating the temperature of the vape device throughout a time period of usage. For example, vaping at lower temperatures may improve flavor as the vaporization temperatures of certain terpenes/terpenoids and flavonoids tend to be lower temperatures than vaporization temperatures of THC or CBD. Smoking generally does not permit as much granular control over the effects or user experience, as combustion temperature is so high (above 230° C./446° F.) that most components of cannabis plants and extracts may be burned off simultaneously without providing a desired effect or experience.

There are many types of vaporizers that can process a cannabis-based vape liquid in different ways (e.g., different temperatures, amounts of ultrasonic energy, or heat exposure times (e.g., residence times) that may be specific to each different vaporizer. Further, physical structures within different vaporizers vary, and this variance may create different flow rates. Some devices allow the user to purchase vaping liquids and fill liquid reservoirs on their own, which may be referred to as “open systems” for vaping substances. Such reservoirs may be part of a cartridge structure, container structure, etc. Where such reservoirs may be dedicated to use with a particular device and/or may be pre-filled, such structures may be referred to as “closed systems.”

With all these variations, there exists a need to provide improved systems and methods of providing a consistent and reliable user vaporization experience. This is especially true given the fact that the composition of materials or concentrates included in vaporizers varies significantly. There is also a need to provide a means for a user of cannabis vaping products to modify vaping temperatures based on vaping conditions of specific concentrates or environmental conditions external to a vaping device. What are also needed are new ways of controlling vaporization temperatures based on one more factors such that particular types of substances may be vaporized according to pre-determined rules or preferences even when conditions vary.

### SUMMARY OF THE PRESENTLY CLAIMED INVENTION

The present disclosure is directed to, methods, apparatus, and non-transitory computer readable storage media that may control the operation of vaporizing substances included in a vaporizer cartridge. A method consistent with the present disclosure may include receiving an identifier that may be used to identify a substance included in a vape cartridge. After the identifier is received it may be sent to an external computing device. The external computing device may then provide data that includes a temperature that can be used to vaporize the substance. Next, the vaporization of the substance may be controlled based on the identified vaporization temperature.

Apparatus consistent with the present disclosure may control the vaporization of substances included in a vaporizer cartridge. Such an apparatus may include a reader that receives a vape cartridge identifier associated with identifying a substance that is included in a vape cartridge. This apparatus may also include a communication interface that sends the vape cartridge identifier to an external computing device and that receives data that identifies a temperature for vaporizing the substance included in the vape cartridge. This apparatus may also include a memory, a temperature sensor, and a processor that executes instructions out of the memory. After data is received from the external computing device, the processor executing instructions out of the memory may receive data from the temperature sensor and control the vaporization of the substance based on vaporization temperature.

When the method of the presently claims invention is implemented as a non-transitory computer readable storage medium, a processor executing instructions out of a memory may perform a method consistent with the present disclosure that controls the vaporization of a substance. Here again, the method may include receiving an identifier that may be used to identify a substance included in a vape cartridge. After the identifier is received it may be sent to an external computing device. The external computing device may then provide data that includes a temperature that can be used to vaporize the substance. Next, the vaporization of the substance may be controlled based on the identified vaporization temperature.

### BRIEF DESCRIPTIONS OF THE DRAWINGS

FIG. 1 illustrates an exemplary network environment in which a system for smart vaporization may be implemented.

FIG. 2 is a flowchart illustrating an exemplary method for identifying materials included in a specific vape cartridge.

FIG. 3 is a flowchart illustrating an exemplary method for a smart vaporizer.

FIG. 4 is a flowchart illustrating an exemplary method for optimizing the temperature of a heating element.

FIG. 5 is a flowchart illustrating an exemplary method for user device communication with a smart vaporizer.

FIG. 6 illustrates an exemplary smart vaporizer that includes a vape body and a vape cartridge.

FIG. 7 illustrates a computing system that may be used to implement an embodiment of the present invention.

### DETAILED DESCRIPTION OF THE DRAWINGS

The present disclosure is directed to apparatus, methods, and non-transitory computer readable storage media that may control the operation of vaporizing cannabinoids included in a vaporizer cartridge. Apparatus and methods consistent with the present disclosure may identify contents included in a vape cartridge based on an identifier that may be scanned or read. Once the materials in a cartridge are identified, a smart vaporizer may be configured to vaporize those materials in controlled ways. In certain instances, recommended vaporization parameters may be modified based on user input. Parameters that control the heating of a heating element or the opening of a valve that controls a flow of liquid from a reservoir at the cartridge may be changed based on environmental conditions such that a user using the smart vaporizer may be able to inhale vaporized substances at preferred temperatures or flow rates even when environmental conditions change.

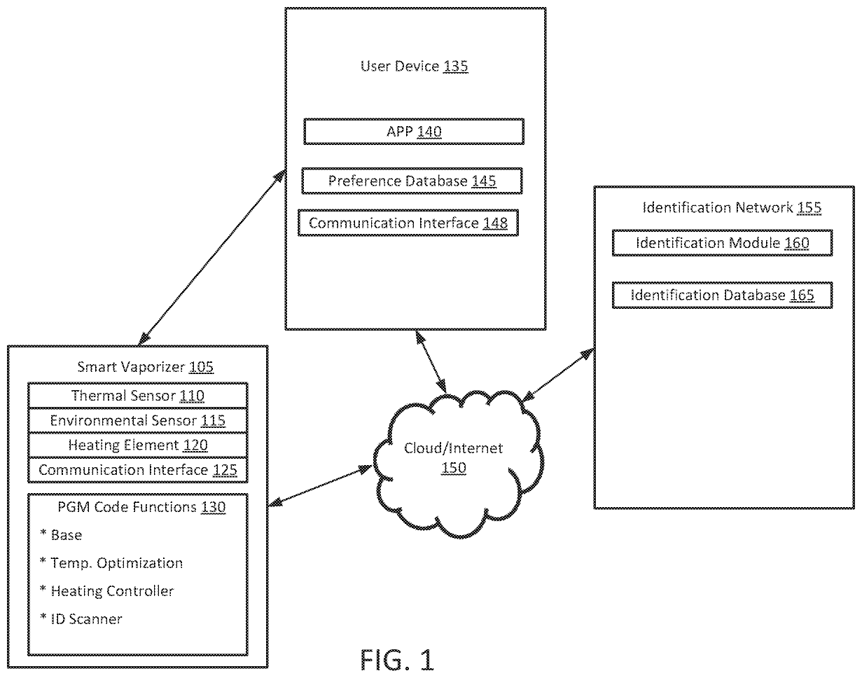

FIG. 1 illustrates an exemplary network environment in which a system for smart vaporization may be implemented. The network environment illustrated in FIG. 1 may include smart vaporizer device 105 that may communicate with user device 135 or identification network server 155 in communication with other device via communications network 150 (e.g., cloud or Internet). Smart vaporizer 105 includes thermal sensor 110, environmental sensor 115, heating element 120, communication interface 125, and program code 130 that may cause a processor to perform certain functions to operate the smart vaporizer 105 according to rules or preferences. Program code functions included in FIG. 1 are base software functions, temperature optimization functions, heating controller functions, and identifier (ID) scanning functions. These functions may be performed by one or more sets of program code that may be implemented as a base software module, a temperature optimization software module, a heating controller module, and an ID scanning module. User device 135 of FIG. 1 includes application program APP 140, preference database 145, and communication interface 148. Identification network 155 includes identification (ID) module 160 and identification (ID) database 165. User device 135 may communicate with smart vaporizer 105 via a short distance wireless communication interface 148 such as an 802.11 Wi-Fi or a Bluetooth interface. Alternatively or additionally smart vaporizer 105, user device 135, and identification network 155 may communicate via the cloud or internet 150 using any communication interface 148 known in the art, including wireless cellular communication interfaces or wired communication connections.

Smart vaporizer 105 may identify a type of material that is included in a cartridge that is connected to smart vaporizer 105. In such instances, a cartridge may include a scan-able identifier or an interface that can provide data to a controller at smart vaporizer 105. In certain instances, smart vaporizer 105 may include a bar code, a quick response (QR) code, radio frequency identifier (RFID), or a near field communication (NFC) tag that includes data that identifies a type of material included in a vape cartridge. Such a code or tag may be scanned by user device 135 that may then communicate with identification network 155 to retrieve data that identifies one or more materials included in the vape cartridge. After receiving the data from identification network 155, user device 135 may store that data and communicate settings or other parameters to smart vaporizer 105. Alternatively, electronics at smart vaporizer 105 may perform the functions of acquiring data from the vape cartridge and retrieving the material identification data from the identification network directly via the cloud or Internet 150 or via user device 135.

The ID module 160 at identification network 155 may be a set of program code executable by a processor that receives vape cartridge information such that data relating to the vape cartridge can be retrieved from ID database 165 or from another database. In certain instances, ID network 155 may retrieve test data from ID database 165 or from another database. Such test data could identify types of cannabinoids included in the cartridge, concentrations of cannabinoids within the cartridge, or information that identifies other substances included in the cartridge. For example, the test data may identify that materials in the cartridge have a THC/CBD ratio of 20/80%, a total cannabinoid content of 200 milligrams (mg), and a filler of propylene glycol. In certain instances, these test results may also identify that the cartridge includes no flavorings, flavorings of certain types, or that may identify that the cartridge does not contain vitamin E acetate. Other information that may be received from ID network 165 include information that identifies a manufacturer, a lot number, a date of manufacture, a recommended temperature, a recommended amount of energy to apply to a heating element, a default stimulus “waveform,” or a default valve setting.

In operation ID module 160 may receive an ID number that uniquely identifies the vape cartridge, and a processor executing instructions out of a memory at ID network 155 may then retrieve related product data and related process data. The ID network 155 may then send this data back to smart vaporizer 105 directly or via user device 135. A processor at user device 135 may execute program code of APP 140 when user device 135 communicates with either ID network 155 or with smart vaporizer 105. Operation of program code at user device 135 may also allow a user to enter personal user preferences that allows them to change recommended or default settings. As such user may identify a preferred temperature, an amount of energy to apply to a heating element, a stimulus “waveform,” or may identify a preferred valve setting. These user settings could allow a user to change default settings to change flavors of inhaled vapors or to adjust a flow rate of material provided to a heating element in smart vaporizer 105. User device may then send recommended, default, or user updated settings to smart vaporizer 105 such that a controller or processor at smart vaporizer may control the operation of smart vaporizer 105 according to those received settings. These settings may also be stored at preference database 145 of user device 135 or these settings may be provided to ID network 155 for storage in ID database 165. As such, vaporizer APP 140 may allow a user of user device 135 to look up the product IDs, modify his/her consumption preferences or connect with other users. Preference database 145 may store any of the data retrieved by user device 135 or may store user preferences set by a user.

As mentioned previously, smart vaporizer 105 may include program code that is executable by processor to perform functions of controlling vaporization parameters according to rules or preferences. Such rules or preferences may be based on recommended, default, or preferred settings. After smart vaporizer 105 receives settings via communication interface 125, a processor at vaporizer 105 may receive sensor data from thermal sensor 110 when applying energy to heating element 120. Environmental sensor 115 may also provide sensor data (e.g. temperature or humidity data) to the processor at vaporizer 105. The sensor data received by the processor may be used to control the temperature of heating element 120. This temperature control may be based on data from thermal sensor 110, from data from environmental sensor 115, or both. In certain instances, on a cold day when air temperature is 35 degrees F., the environmental sensor data may allow the processor to increase an amount of energy provided to heating element 120, such that smart vaporizer 105 can actually provide heated vapor to a user that matches a temperature setting.

Program code functions 130 may be stored in a memory at smart vaporizer 105 when a processor executes instructions that perform a set of base functions, one or more temperature optimization functions, a heating control function, or when an identifier of vaporized materials is evaluated. These different functions may be implemented as one or more sets of program code. In one instance, program code functions 130 may be implemented by a base software module, a temperature optimization software module, a heating controller module, and an scanner identification module. Program code functions 130 may allow vaporizer 105 to operate according to settings as described above. Operation of program code functions 130 may allow a heating control program module to receive instructions from a temperature optimization program module. The temperature optimization module may receive information that identifies a concentrate included in a vape cartridge, this temperature optimization module may adjust a temperature of heating element 120 or an amount of energy provided to heating element 120 according to a set of pre-determined conditions or settings. In some embodiments, ambient conditions or user preferences may be incorporated into a calculation used to determine an optimized heating procedure when controlling operation of the heating element 120. Heating controller program code may also be used to control a vaporization temperature based upon product requirements or user preferences. In certain instances, these requirements or preferences may cause a processor executing a set of heating controller program code to adjust a temperature of heating element 120 as environmental conditions at the vaporizer device 105 may change.

In certain instances, a scanner or an electrical interface at vaporizer 105 may provide data to a processor at vaporizer 105 when the processor checks to see whether a vape cartridge connected to vaporizer 105 contains an expected set of materials. In an instance where a user inadvertently connects an incorrect cartridge to vaporizer 105, the processor may identify from scanned data that the attached cartridge includes an incorrect THC to CBD ratio. For example, when vaporizer 105 is configured to vaporize a concentrate that has a THC to CBD ratio of 10% to 90%, yet identifies that the attached cartridge includes a concentrate with a THC to CBD ratio of 95% to 5%, the processor could initiate an alarm and disable the heating element of vaporizer 105. At this time the processor at smart vaporizer 105 may provide a warning to a user directly or via communication sent to user device 135. Such alarms or warnings may help prevent a user from inhaling concentrations of cannabinoids that are not consistent with their preferences or with a current set of vape settings.

In certain instances, preferences may be provided via the cloud or Internet 150 to identification network 155 when a user provides a user profile and that may include preferred conditions of vaporization. A user may also register their device, preferred cartridge identifiers, or preferred settings. After such a registration process, a user may connect a cartridge to vaporizer 105, depress a start (ignition) button that causes heating element 120 to heat, and the user may then inhale vapors according to settings consistent with user preferences, recommended settings, or default settings. Even in instances, when a person walks from a warm room into a freezer, and then presses the start (ignition) button, smart vaporizer 105 may adjust an amount of energy provided to heating element 120 such that a same end-product is provided to the user despite a change in ambient temperature. Since repeated heating of a heating element may cause the heating element to stay hot, a current sensed temperature of a heating element may cause program code to change an amount of energy provided to the heating element the next time the start button is depressed. This may prevent overheating of the heating element by repeated application of the start button and so may prevent inadvertent inhalation of undesirable compounds such as degradation products of the cannabinoids or diluents present in the vape liquid.

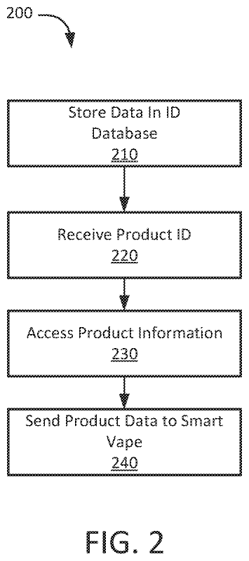

FIG. 2 illustrates an exemplary method 200 for identifying materials included in a specific vape cartridge. The method of FIG. 2 may also allow a networked computer system to provide recommended settings for controlling the vaporization of materials inside of a specific vape cartridge based on a received product identifier. The method 200 of FIG. 2 begins with step 210 where data may be stored in a database, such as ID database 165 of FIG. 1. The data stored in the ID database in step 210 may store vape cartridge type identifiers from which materials included in a vape cartridge (e.g. THC/CBD ratio), a manufacturer, a lot number, a date of manufacture, a recommended temperature, a recommended amount of energy to apply to a heating element, a default stimulus “waveform,” or a default valve setting may be identified. Next, in step 220 of FIG. 2 a product identifier may be received. Here again this product identifier may uniquely identify that a vape cartridge includes specific types and concentrations of materials. This identifier may have been received via a communication from a user device or may have been received in a communication directly from a smart device. From this identifier, product information may be retrieved from an ID database in step 230 of FIG. 2 and that information may be provided to smart vape either directly or via a user device in step 240 of FIG. 2. This process may allow smart vaporizer 105 of FIG. 1 to receive settings that may be used to vaporize materials according to a set of recommended or default settings.

TABLE 1

| Product | THC/CBD | Temp | | Date of |
|---|---|---|---|---|
| ID | Ratio | Range (° F.) | MFG | MFG |
| P105 | 25/75 | 350-365 | ABC | Jan. 1, 2018 |
| P221 | 20/80 | 330-355 | ABC | Feb. 1, 2018 |
| P313 | 10/90 | 330-340 | Radient | Mar. 1, 2018 |
| P004 | 50/50 | 350-355 | Radient | Apr. 1, 2018 |
| . . . | . . . | . . . | . . . | . . . |
| . . . | . . . | . . . | . . . | . . . |
| P505 | 95/5 | 321-355 | ABC | Jul. 1, 2018 |

Table 1 includes an exemplary set of information that may be stored in a database at a user device, such as the preference database 145 of user device 135 FIG. 1. Table 1 includes columns of information that include product identifiers (ID), THC/CBD ratios, temperature ranges, manufacturers, and a date of manufacturer. Note that product ID P105 includes a THC/CBD ratio of 25% THC to 75% CBD (25/75), a temperature range of 350-360 degrees F., a manufacturer ABC, and a date of manufacture of Jan. 1, 2018. Note also that product ID P004 includes a THC/CBD ratio of 50% THC to 50% CBD, a temperature range of 350-355 degrees F., a manufacturer Radient, and a date of manufacture of Apr. 1, 2018. The data of table 1 cross-references product identifiers with specific material compositions, and recommended temperature ranges for vaporizing materials included in vape canisters manufactured by specific manufacturers. Since the contents of table 1 may be stored at a user device, this user device may be able to send vaporization temperatures to a smart vaporizer whenever a cartridge with a different product identifier included in table 1 is connected to the smart vaporizer. Here again, such settings may be sent to smart vaporizer via a wireless communication.

In certain instances, the parameters included in table 1 may change or be changed based upon the requirements or recommendations of a specific product or based on user preferences. For instance, P221 may need a different energy for a piezo-electric controller vs. product P313. For instance, P221 may need a different amount of time in the residence chamber vs. product 313. For instance, P221 may need a different valve adjustment for inhaling vs. product P313. For instance, P221 may need a different pulsing waveform to optimize the conversion of active ingredients to actives vs. product P313. There are many varied “other parameters” that may need to be changed or adjusted on the smart device. It is also likely that a number of these parameters may have been optimized in a multivariable optimization, so each product ID would drive various smart devices process parameters, for pre-tested different environmental factors (temperature, humidity etc.).

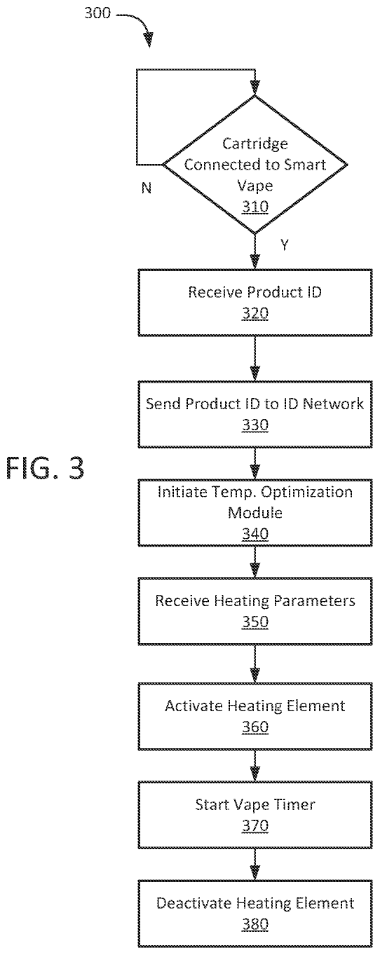

FIG. 3 is a flowchart illustrating an exemplary method for a smart vaporizer. The method 300 of FIG. 3 begins with determination step 310 that identifies whether a cartridge is attached to a smart vape, when no, program flow may move back to step 310. In certain instances, step 310 may only be evaluated when an start or ignition switch is depressed at the smart vape. When a cartridge is attached to smart vape 310, program flow may move to step 320 where a product identifier (ID) is received. The product ID received in step 320 may be received by a scanner at the smart vape or may be identified using data communicated via a direct electrical connection that retrieves the product ID from a storage element at the cartridge. After step 320, the product ID may be sent to an identification network, such as the ID network 155 of FIG. 1. Here again this product ID may be sent directly to the ID network from the smart vape or may be forwarded via (through) a user device to the ID network.

Step 340 of FIG. 3 is a step that may initiate a program code consistent with a temperature optimization module that causes a processor at the smart vape to control heating of a heating element at a smart vape. Next, in step 350 heating parameters may be received and the heating element may be energized based on operation of the temperature optimization program code. Temperature optimization may be performed by applying a voltage to a heating element, applying a current to the heating element, pulsing a voltage or current applied to the heating limit, or changing a voltage or current applied to the heating element in controlled ways. The pulsing or changing of voltages or currents applied to a heating element may correspond to a “waveform” setting that may cause the heating element to reach a desired temperature in a predetermined amount of time, without exceeding the desired temperature. When the heating element is initially energized, it may be energized with a higher voltage or current and that voltage or current may be reduced after a period of time. This could cause the heating element to rapidly heat over a first time period (e.g. 250 milliseconds) and then to be gently heated after that first time period. This could cause the heating element to rapidly heat from an ambient or current temperature to a temperature of 320 degrees Fahrenheit (F) over the first time period and could cause the heating element temperature to not exceed 350 degrees F. Rapid heating followed by more gentle heating may also be controlled by applying a voltage or current over a first time period and then by pulsing the voltage or current at intervals consistent with a profile characteristic of the heating element. Step 370 of FIG. 3 is a step where a timer may be started at a smart vape, this timer may be used to either set a maximum time that the heating element may be energized after a start switch is depressed at a smart vape or may be used to monitor times when energy is provided to the heating element. As such, the timer of step 370 may be used to control changing voltages or currents applied to a heating element or may be used to pulse voltages or currents provided to a heating element. Next, the heating element in the smart vape may be deactivated (de-energized or turned off) in step 380 of FIG. 3.

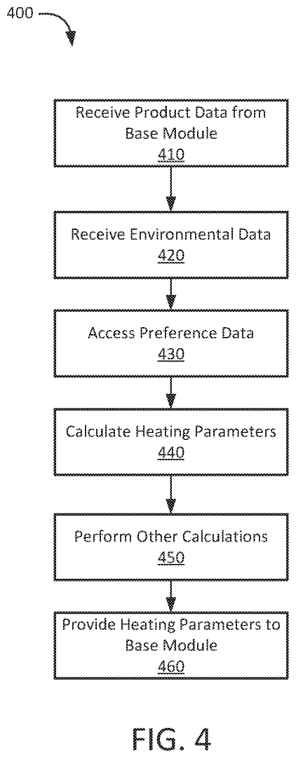

FIG. 4 is a flowchart illustrating an exemplary method for optimizing the temperature of a heating element. The flow chart 400 of FIG. 4 begins with step 410 where information may be received from a set of base program code such as the program code of FIG. 3. The information received may identify a product or material that is included in a vape cartridge attached to a smart vape. Next, in step 420 environmental data may be received (e.g. an ambient temperature) and then preference data may be accessed in step 430 of FIG. 4. A calculation may then be performed in step 440 that identifies heating parameters that should be used to control the heating of a heating element of a vape cartridge based on changes associated with the environmental data. The calculations performed in step 440 may cause a heating element to be energized more or less aggressively when a user is located in either a cold or a hot environment. A more aggressive energizing of a heating element may correspond to an increased voltage or current applied to a heating element. Alternatively, a more aggressive heating of the heating element may correspond to increased amounts of time that energy is provided to the heating element in each of a series of pulses. Less aggressive energization of a heating element may correspond a reduction of voltage, current, or pulsewidth times applied to the heating element. The pulsing of voltages or currents to a heating element may correspond to waveforms that have “on” or “energized” times and “off” or “de-energized” times according to a pulsewidth modulation profile that reduces amounts of time that energy is provided to a heating element as a temperature of the heating element approaches a desired temperature. The calculations performed in step 440 of FIG. 4 may cause a set of standard heating parameters to be changed when the vape is in a cold or in a hot environment such that the user can inhale vapors at a preferred temperature or flow rate even when environmental conditions change.

Next, in step 450 of FIG. 4 other calculations may be performed that may identify that a valve setting should be adjusted based on environmental data such that a flow rate of vapor can be maintained given cold or hot environmental data. For example, a cold temperature may cause baseline valve setting to be open the valve to a greater size when the temperature is below a cold threshold level. Alternatively, a temperature above a hot threshold level may result in the valve being closed as compared to a baseline valve setting. These valve changes may correspond to the fact that a material or liquid included in a cartridge may become more viscous in cold temperatures and may become less viscous in high temperatures. An environmental temperature that is too cold or too hot may cause the material to flow too slowly or too quickly in cold or hot conditions unless a valve setting is changed. After step 450, heating or other parameters may be provided to a base software module that controls the heating or valve settings of the smart vape in step 460 of FIG. 4.

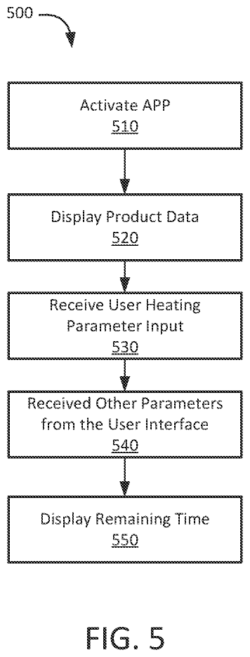

FIG. 5 is a flowchart illustrating an exemplary method 500 for user device communications with a smart vaporizer. The method 500 of FIG. 5 may be consistent with actions performed by application program (APP) 140 of FIG. 1. Step 510 of FIG. 1 may be a step where a set of application program code at a user device is activated (started or initiated). This may be after a user selects an application program in a user interface at their user device. The application program code at the user device may have previously been downloaded from an application store like the Apple APP store and functionality of this application program may extend capabilities of a user device to be capable of communicating with a smart vape. Next, in step 520 product data may be displayed on a display of a user device. In certain instances, this display may include a graphical user interface (GUI) that a user may interact with when making selections or when inputting information. User device 135 of FIG. 1 may receive data that identifies material included in a vape cartridge. Here again, this data may have been received from a smart vape or may have been received by scanning an identifier of the vape cartridge. User device may then have accessed a local database or may collect product data from an external computer, such as ID network computer 155 of FIG. 1. Once the product data is received in step 520, the user device may receive heating inputs from a user via the GUI at the user device. The user input received may be an indication that the heating of the heating element should be decreased by 10% or that a valve at the smart vape should be increased by 20%, for example. Other parameters may also be received via the GUI in step 540 of FIG. 5. These other parameters may identify a heating waveform selection or may include threshold temperatures (e.g. a low or high temperature threshold) for adjusting heating or valve opening parameters at different ambient temperatures. The application program code at the user device may allow a user to try different settings and then select a preferred setting to use for a given type of material included vape cartridges that are identified by particular vape cartridge identifiers.

TABLE 2

| | | | Preferred | | | |
|---|---|---|---|---|---|---|
| User | User | | Temp. | | | Valve |
| ID | Name | Age | (° F.) | Energy | Waveform | Setting |
| 667 | Apple | 37 | 321 | Normal | W21 | Normal |
| 603 | Orchid | 27 | 350 | Normal | W33 | Normal −10% |
| 604 | Rye | 35 | 381 | Normal −20% | W31 | Normal |
| 605 | Pink | 21 | 340 | Normal | W33 | Normal |
| 605 | Beige | 22 | 361 | Normal | W41 | Normal +20% |
| 607 | Cherry | 27 | 362 | Normal +20% | W45 | Normal |
| 608 | Oyster | 25 | 322 | Normal | W12 | Normal |
| 609 | Ocean | 25 | 350 | Normal | W12 | Normal −10% |
| 610 | Cyean | 26 | 350 | Normal | W33 | Normal |
| 611 | Plum | 65 | 361 | Normal | W31 | Normal |
| 612 | Rose | 44 | 382 | Normal | W54 | Normal |

Table 2 includes an exemplary set of information may be stored in a database that cross-references user identifiers with user data or user preferences. The data illustrated in table 2 may be stored in ID database 165 of FIG. 1. Columns of table 2 include user identifiers, user names, ages of respective users, preferred user vaporization temperatures, energy levels that may be provided to a heating element, waveforms of energy stimulus that may be provided to a heating element, and valve settings. These different sets of information may have been received from different user devices after user those respective user devices have selected settings that they prefer. Each respective row in table 2 corresponds to a different user ID and a different user name. Note that user ID 667 corresponds to user name Apple that belongs to a 37 year old person that prefers a vape temperature of 321 degrees F. User ID 667 has selected a normal energy, a waveform stimulus of W21, and a normal valve setting. Note that user ID 604 prefers a vape temperature of 381 degrees F., an energy of normal minus 20%, a wave form of W31, and a normal valve settings. Note also that one energy setting in table 2 is set at normal plus 20% and that other valve settings in table 2 include normal minus 10% and normal plus 20%.

An amount of energy may correspond to a voltage (E), a current (I), or a power (P) in Watts that may be calculated by the formula P=I*E. Energy amounts may also be identified based on lengths of times that a voltage or a current is provided to a heating element, where a total amount of power provided to a heating element over a period of time may be proportional to energized times over a span of time.

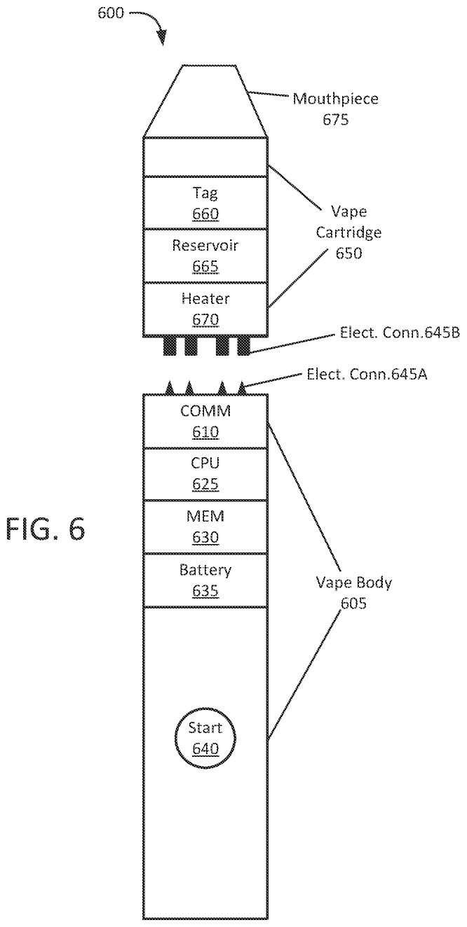

FIG. 6 illustrates an exemplary smart vaporizer that includes a vape body and a vape cartridge consistent with the present disclosure. The smart vaporizer 600 of FIG. 6 includes vape body 605 and vape cartridge 650. The vape body 605 includes a communication interface (COMM) 610, a processor (CPU) 610, a memory (MEM) 625, battery 635, and a start button 640, and electrical connectors 645A. Note that the COMM 610 may include a scanner capable of receiving an image of a bar code or a QR code. Alternatively COMM 610 may include a scanner capable of scanning a RFID or an NFC chip that may be attached to or that may be built into vape cartridge 650. COMM 610 may additionally include a wireless communication interface, such as a Wi-Fi, Bluetooth, or a cellular interface that is capable of communicating with external computing devices. Vape cartridge 650 includes electrical connectors 645B, tag 660, reservoir 665, heater 670, and mouthpiece 675. Note that electrical connectors 645A and 645B may be used to provide power from battery 635 to heater 670 or may be used to read data stored in a memory at vape cartridge 650. As such, the CPU 625 may receive an identifier from vape cartridge by reading a bar code or QR code, by scanning an RFID or NFC chip, or by reading data from a memory at vape cartridge 650 using COMM 610 or electrical connectors 645A & 645B.

Since the smart vaporizer 600 of FIG. 6 includes processor 625, memory 630, battery 635, communication interface 610, and electrical connectors 645A/B, CPU 625 may perform operations consistent with program code functions 130 of FIG. 1, the base functions of FIG. 3, or the environmental optimizations of FIG. 4. As such, when a user presses start button 640 the heater (heating element) 670 may be energized according to preferred energization parameters when vaporizing cannabinoids or other materials included in reservoir 665. After start button 640 is depressed and materials from reservoir 665 are vaporized, a person sucking on mouthpiece 675 may inhale the vaporized materials according to predetermined settings or preferences.

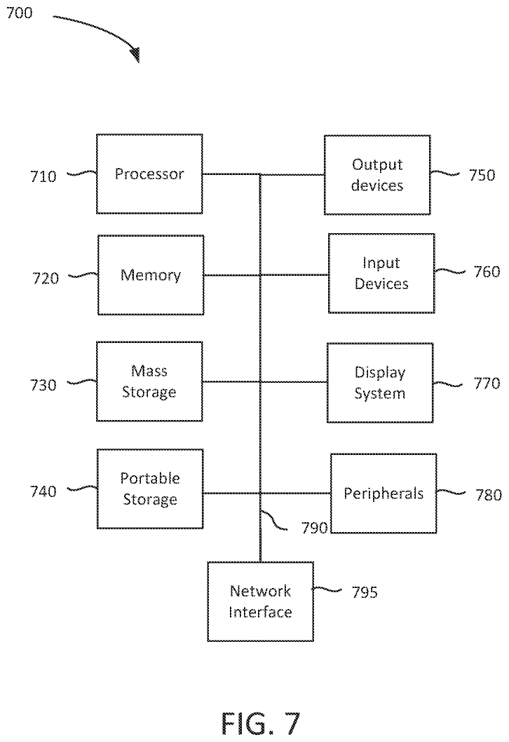

FIG. 7 illustrates a computing system that may be used to implement an embodiment of the present invention. The computing system 700 of FIG. 7 includes one or more processors 710 and main memory 720. Main memory 720 stores, in part, instructions and data for execution by processor 710. Main memory 720 can store the executable code when in operation. The system 700 of FIG. 7 further includes a mass storage device 730, portable storage medium drive(s) 740, output devices 750, user input devices 760, a graphics display 770, peripheral devices 780, and network interface 795.

The components shown in FIG. 7 are depicted as being connected via a single bus 790. However, the components may be connected through one or more data transport means. For example, processor unit 710 and main memory 720 may be connected via a local microprocessor bus, and the mass storage device 730, peripheral device(s) 780, portable storage device 740, and display system 770 may be connected via one or more input/output (I/O) buses.

Mass storage device 730, which may be implemented with a magnetic disk drive or an optical disk drive, is a non-volatile storage device for storing data and instructions for use by processor unit 710. Mass storage device 730 can store the system software for implementing embodiments of the present invention for purposes of loading that software into main memory 720.

Portable storage device 740 operates in conjunction with a portable non-volatile storage medium, such as a FLASH memory, compact disk or Digital video disc, to input and output data and code to and from the computer system 700 of FIG. 7. The system software for implementing embodiments of the present invention may be stored on such a portable medium and input to the computer system 700 via the portable storage device 740.

Input devices 760 provide a portion of a user interface. Input devices 760 may include an alpha-numeric keypad, such as a keyboard, for inputting alpha-numeric and other information, or a pointing device, such as a mouse, a trackball, stylus, or cursor direction keys. Additionally, the system 700 as shown in FIG. 7 includes output devices 750. Examples of suitable output devices include speakers, printers, network interfaces, and monitors.

Display system 770 may include a liquid crystal display (LCD), a plasma display, an organic light-emitting diode (OLED) display, an electronic ink display, a projector-based display, a holographic display, or another suitable display device. Display system 770 receives textual and graphical information, and processes the information for output to the display device. The display system 770 may include multiple-touch touchscreen input capabilities, such as capacitive touch detection, resistive touch detection, surface acoustic wave touch detection, or infrared touch detection. Such touchscreen input capabilities may or may not allow for variable pressure or force detection.

Peripherals 780 may include any type of computer support device to add additional functionality to the computer system. For example, peripheral device(s) 780 may include a modem or a router.

Network interface 795 may include any form of computer interface of a computer, whether that be a wired network or a wireless interface. As such, network interface 795 may be an Ethernet network interface, a BlueTooth™ wireless interface, an 802.11 interface, or a cellular phone interface.

The components contained in the computer system 700 of FIG. 7 are those typically found in computer systems that may be suitable for use with embodiments of the present invention and are intended to represent a broad category of such computer components that are well known in the art. Thus, the computer system 700 of FIG. 7 can be a personal computer, a hand held computing device, a telephone (“smart” or otherwise), a mobile computing device, a workstation, a server (on a server rack or otherwise), a minicomputer, a mainframe computer, a tablet computing device, a wearable device (such as a watch, a ring, a pair of glasses, or another type of jewelry/clothing/accessory), a video game console (portable or otherwise), an e-book reader, a media player device (portable or otherwise), a vehicle-based computer, some combination thereof, or any other computing device. The computer can also include different bus configurations, networked platforms, multi-processor platforms, etc. The computer system 700 may in some cases be a virtual computer system executed by another computer system. Various operating systems can be used including Unix, Linux, Windows, Macintosh OS, Palm OS, Android, iOS, and other suitable operating systems.

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASH EPROM, and any other memory chip or cartridge.

The accompanying drawings illustrate various embodiments of systems, methods, and embodiments of various other aspects of the disclosure. Any person with ordinary skills in the art may appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

While various flow diagrams provided and described above may show a particular order of operations performed by certain embodiments of the invention, it should be understood that such order is exemplary (e.g., alternative embodiments can perform the operations in a different order, combine certain operations, overlap certain operations, etc.).

What is claimed is:

1. A method for cartridge-specific vaporization control, the method comprising:
receiving an identifier associated with a vape cartridge;
sending the identifier to a computing device, wherein the computing device stores information associated with the identifier including information regarding at least one substance included in the vape cartridge;
receiving the information associated with the identifier sent from the computing device, wherein the received information indicates one or more vaporization settings recommended for vaporizing the at least one substance; and
controlling vaporization from the vape cartridge by a vape device based on the recommended vaporization settings.

2. The method of claim 1, further comprising identifying a recommended vaporization temperature based on the vaporization settings and providing the recommended vaporization temperature to the vape device.

3. The method of claim 1, further comprising receiving a user preference specifying a change to at least one of the recommended vaporization settings, wherein the vaporization from the vape cartridge is controlled further based on the change specified by the user preference.

4. The method of claim 3, storing the user preference in memory.

5. The method of claim 1, wherein controlling the vaporization from the vape cartridge comprises:
identifying that a vaporization switch of the vape device has been pressed; and
controlling energy provided to a heating element of the vape device, wherein the energy is controlled in accordance with the recommended vaporization settings.

6. The method of claim 5, wherein controlling the energy includes providing a specified level of at least one of a voltage or a current to the heating element.

7. The method of claim 6, wherein the recommended vaporization settings includes a waveform setting specifying a set of time periods during which the specified level is provided, and wherein changing the at least one of the voltage or the current is based on the waveform setting.

8. The method of claim 1, further comprising:
receiving at least one of a temperature or a humidity from an environmental sensor; and
identifying a current environmental condition based on the received information from the environmental sensor, wherein controlling the vaporization is further based on the identified current environmental condition.

9. The method of claim 1, wherein the recommended vaporization settings includes a valve setting for a valve of a reservoir holding the at least one substance within the vape cartridge, and wherein controlling the vaporization includes opening the valve in accordance with the valve setting.

10. An apparatus for cartridge-specific vaporization control, the apparatus comprising:
a reader that receives an identifier associated with a vape cartridge;
a communication interface that communicates over a communication network to:
send the identifier to a computing device, wherein the computing device stores information associated with the identifier including information regarding at least one substance included in the vape cartridge, and
receive the information associated with the identifier sent from the computing device, wherein the received information indicates one or more vaporization settings recommended for vaporizing the at least one substance from the vape cartridge; and
a processor that executes instructions stored in memory, wherein the processor executes the instructions to control vaporization from the vape cartridge based on the recommended vaporization settings.

11. The apparatus of claim 10, wherein the processor further identifies a recommended vaporization temperature based on the vaporization settings and provides the recommended vaporization temperature to the vape device

12. The apparatus of claim 10, further comprising a user interface that receives a user preference specifying a change to at least one of the recommended vaporization settings, wherein the vaporization from the vape cartridge is controlled further based on the change specified by the user preference.

13. The apparatus of claim 12, further comprising a memory that stores the user preference.

14. The apparatus of claim 10, further comprising:
a battery that provides an amount of energy;
a heating element that generates heat based on the amount of energy provided from the battery, and
a vaporization switch that is pressed, wherein the processor controls the vaporization from the vape cartridge when the vaporization switch has been pressed by controlling the amount of energy provided from the batter to the heating element, wherein the amount of energy is controlled in accordance with the recommended vaporization settings.

15. The apparatus of claim 14, wherein the processor controls the amount of energy by specifying a level of at least one of a voltage or a current to the heating element.

16. The apparatus of claim 15, wherein the recommended vaporization settings includes a waveform setting specifying a set of time periods during which the specified level is provided, and wherein the processor specifies changing the at least one of the voltage or the current based on the waveform setting.

17. The apparatus of claim 10, further comprising an environment sensor that detects at least one of a temperature or a humidity, wherein the processor further identifies a current environmental condition based on the received information from the environmental sensor, and controls the vaporization further based on the identified current environmental condition.

18. The apparatus of claim 10, wherein the recommended vaporization settings includes a valve setting for a valve of a reservoir holding the at least one substance within the vape cartridge, and wherein the processor controls the vaporization by specifying that the valve is to be opened in accordance with the valve setting.

19. A non-transitory, computer-readable storage medium having embodied thereon a program executable by a processor for performing a method for cartridge-specific vaporization control, the method comprising:
receiving an identifier associated with a vape cartridge;
sending the identifier to a computing device, wherein the computing device stores information associated with the identifier including information regarding at least one substance included in the vape cartridge;
receiving the information associated with the identifier sent from the computing device, wherein the received information indicates one or more vaporization settings recommended for vaporizing the at least one substance; and
controlling vaporization from the vape cartridge by a vape device based on the recommended vaporization settings.
